(19) United States
(12) Patent Application Publication
(10) Pub. No.: US 2024/0341765 A1
(43) Pub. Date: Oct. 17, 2024

(54) NERVE COUPLER AND METHOD FOR USE OF THE SAME

(71) Applicants: Mary Hitchcock Memorial Hospital, for itself and on behalf of Dartmouth-Hitchcock Clinic, Lebanon, NH (US); THE TRUSTEES OF THE UNIVERSITY OF PENNSYLVANIA, Philadelphia, PA (US); The United States Government as represented by the Department of Veterans Affairs, Washington, DC (US)

(72) Inventors: Joseph M. Rosen, Lyme, NH (US); Daniel Kacy Cullen, Media, PA (US); Justin C. Burrell, Philadelphia, PA (US); Basile Montagnese, Ontario (CA); Theodore Bronk, Woodside, CA (US); Joshua De La Cruz, Elmhurst, IL (US); Gracyn Campbell, Larchmont, NY (US); Yutong Xia, Providence, RI (US); Reeham M. Choudhury, Milford, MA (US)

(21) Appl. No.: 18/294,467

(22) PCT Filed: Aug. 2, 2022
(86) PCT No.: PCT/US22/39190

§ 371 Date: Feb. 1, 2024

Related U.S. Application Data
(60) Provisional application No. 63/228,308, filed on Aug. 2, 2021.

(51) Int. Cl. A61B 17/11; A61B 17/00

(52) U.S. Cl. CPC A61B 17/1128; A61B 2017/00004; A61B 2017/00022; A61B 2017/00477; A61B 2017/1132; A61B 2217/007

(57) ABSTRACT

A system and method for repairing a damaged peripheral nerve is provided. A coupler includes a stabilizing assembly applied to a distal end of a damaged area of the nerve and applied to a proximal end of the damaged area, the coupler adapted so that the proximal end and the distal end are maintained in a stabile abutting relationship in which nerve repair can occur. A chamber is placed in proximity to the damaged area, having a therapeutic agent. Illustratively, the distal end and the proximal end are severed from each other. The coupler can define one of (a) coupler halves that surround the proximal end and the distal end and allow fixing members to be applied to the proximal end and the distal end and (b) cuffs that engage, and are fixed to, each of the proximal end and the distal end and are engaged by overlying coupler halves.

### STATEMENT REGARDING FEDERALLY SPONSORED RESEARCH OR DEVELOPMENT

The inventor, Dr. Joseph M. Rosen has been supported during his career by VA Merit Award Funding.

### FIELD OF THE INVENTION

This invention relates to repair of nerve damage and more particularly to systems and methods for coupling severed ends of nerve bundles to facilitate regeneration of connections therebetween.

### BACKGROUND OF THE INVENTION

Nerve-coupling devices have been known and in use to repair damaged nerves for several decades. In general, couplers are designed to pass over each end of a severed a peripheral nerve and associated axons, and then to use sutures or other mechanical mechanisms to draw then ends together so that they are in alignment and closely adjacent. The ends are then allowed to grow linking nerve cells in an attempt to reconnect the two ends in a manner that allows passage of nerve impulses, and at least, partial restoration of nerve function. To facilitate such growth, various fusogens are applied to the nerves, such as polyethylene glycol (PEG). A technique and associated coupler are described in Deirdre M. Marshall, MD, Morton Grosser, PhD, Michael C. Stephanides, Robert D. Keeley, and Joseph M. Rosen, MD, Sutureless nerve repair at the fascicular level using a nerve coupler, Journal of Rehabilitation Research and Development (U.S. Veterans Administration), Vol. 26 No. 3, Pages 63-76 (Summer 1989), which is incorporated herein by reference as useful background information. Related research at Stanford University in the 1980s was undertaken by the inventor herein to create a brain-machine interface involving a computer chip with perforations that allow for an interface between the two ends of the peripheral nerve for axons to grow through it, and thereby create a connection between the axons and the chip.

More particularly, current techniques for nerve repair employ micro sutures that attach the nerve ends together and/or cuffs can be employed to assist in connecting severed nerve ends. Present research and recent clinical studies have revealed that, if a peripheral nerve is severed, and then its axons are brought together in relatively close proximity in a stable manner (i.e. free of motion therebetween), the axons can be fused relatively immediately with the application of a plurality of different compounds that are commonly termed “fusogens”. As described above, PEG is one such fusogen and the term should be taken broadly to include any biocompatible, chemical compound, or mixture of compounds, that facilitates fusion of severed nerve ends, and otherwise avoids the effects of Wallerian degeneration, while allowing for repair of an injury to the nerve. By essentially bathing the nerve ends in fusogen, an immediate fusion can be achieved across nerve ends. While PEG is one exemplary fusogen, this list can further include, but not be limited to chitosan, dextran sulfate, n-nonyl bromide, calcium, sodium nitrate, and H-α-7. Appropriate solvents, such as saline can be mixed with the fusogen(s) to provide an appropriate concentration with which to bathe the damaged region of the nerve.

However, present methods for delivering fusogens and retaining nerve ends in apposition are poorly suited to allow for positional stability while fusogens are applied, and thus, the desired fusion of large numbers of axons together across the nerve ends. Rather, the present approach, which relies mainly on micro sutures to attach nerve ends provides poor apposition of such ends and of the underlying axons that must be fused. As such only partial (and very limited) fusion may occur, or it may fail completely.

In general, if peripheral nerves can be effectively fused, then the normal process of Wallerian degeneration can be avoided. This process begins when the nerve is cut, and the distal axons at the cut undergo associated degeneration. Wallerian degeneration is a typical result of a crushing or cutting injury to a nerve, and once such degeneration begins, the distal axon is lost and the nerve must now regrow from the repair site down the distal nerve axon at a rate of approximately one millimeter per day (approximately one inch per month). As the repair process can take 12 to 18 months or longer, distal end organs, such as muscles, can become atrophied and permanently disabled in the time it takes for nerve regeneration to complete under these conditions.

There are close to 500,000 nerve repairs performed each year in the U.S., and the loss of economic activity due to resulting disabilities can number in the billions of dollars annually. As many nerve injuries occur in military personnel as a result of service-related causes, the ability to provide a rapid and effective nerve repair technique is a high priority for government entities and the defense establishment. Along with these 500,000 nerve injuries, there are more than five million painful nerves and nerve compressions each year in America—for example nerve injuries from surgeries performed by general surgery OBGYN, neurosurgery, dermatological surgeries, orthopedic surgery, podiatry, plastic surgery, hand surgery, thoracic surgery, breast surgery, GI surgery, as well as hand surgeries and peripheral surgeries that are performed by (among others) general surgeons, neurosurgeons, orthopedic surgeons and plastic surgeons. Once pain and compression have occurred, many of these conditions fail conservative therapy with medications and nerve blocks.

Attempts to interconnect severed or damaged nerve endings are taught in the art. One such approach is disclosed in U.S. Pat. No. 9,808,616, entitled REGENERATIVE PERIPHERAL NERVE INTERFACE, issued Nov. 7, 2017, by Paul S. Cederna, et al. This approach employs an insulating substrate, with at least one metallic electrode deposited onto the insulating substrate to form a thin-film array. A portion of the at least one metallic electrode surface contains a layer of a first conductive polymer and a layer of decellularized small intestinal submucosa (SIS) coating a portion of the electrode. A second conductive polymer is electrochemically polymerized through the SIS to form the regenerative peripheral nerve interface. However, this approach does not accommodate physical and functional reconnection of one side of a severed nerve directly to another side thereof.

Thus, it is highly desirable to provide a system and method for nerve repair that takes advantage of the benefits in immediate fusion, and reduction of Wallerian degeneration, that can occur in a treatment environment that provides for stable apposition of nerve ends and continuous/copious application of fusogens and other chemical compounds to the treatment site-in a manner that current procedures using micro sutures and/or cuffs do not.

### SUMMARY OF THE INVENTION

This invention overcomes disadvantages of the prior art by providing a system and method, and associated nerve coupler/device, for repairing severed peripheral nerves that uniquely allows fusion of nerves with the two severed ends in closely confronting and stable apposition to each other and in a manner that delivers appropriate chemical compounds (e.g. fusogens) to the repair site in a controlled manner for an appropriate period of time. The introduction of fusogens (such as PEG) in this manner can avoid losses of nerve axons due to the cascading effects that cause Wallerian degeneration, and allow for substantially immediate fusion of the nerves in each end. Hence the patient can more fully and quickly recover substantially full function. That is the coupler/device effectively blocks apoptosis or cell death so that wallerian degeneration and retrograde degeneration are stopped, and the axon remains alive or in essence immortal. Moreover the nerve coupler/device contemplated herein effectively addresses pain caused by surgery, compression, etc. in that it can act as a nerve pain stimulator via delivery of electrical signals and/or compounds to the underlying nerve. The coupler/device herein effectively overcomes challenges in applying such a device to small nerves in a stable way. Hence, it allows an associated nerve stimulation generator to turn off the nerve and avoid narcotic therapy or other modalities. This arrangement also effectively avoids a need for an invasive spinal cord stimulator to achieve pain control.

In an illustrative embodiment, a system and method for repairing a damaged peripheral nerve is provided. It includes a coupler defining a first coupler half and a second coupler half divided along a longitudinal plane having an inner lumen that is sized and arranged to receive each side of the peripheral nerve at a transected location thereof, the coupler including slots for receiving sutures that anchor each side in an abutting orientation. A chamber is adapted to surround a portion of the coupler so as to bathe the transected location in a therapeutic agent. Illustratively, the coupler includes a slot and bottom support located centrally along the longitudinal plane constructed an arranged to facilitate transection of the peripheral nerve. The bottom support is arranged to maintain the coupler free of flexure. The first coupler half and the second coupler half can each include interengaging, friction fit tabs and slots for removably securing one to the other. The first coupler half and the second coupler half can be substantially identical in size and shape. The coupler or the chamber can be constructed from at least one of permanent and resorbable materials. An external source of the therapeutic agent can be provided, which flows to the chamber at a predetermined rate, and a flow controller can be employed to meter the rate. The therapeutic agent can be a fusogen, such as, at least one of PEG, chitosan, dextran sulfate, n-nonyl bromide, calcium, sodium nitrate, and H-α-7. The first chamber half and the second chamber half can each include interengaging, friction fit tabs and slots for removably securing one to the other. A lid can be provided, which is adapted to nest within inner walls of the chamber and surround the coupler. The first chamber half and the second chamber half can be substantially identical in size and shape, so as to alleviate confusion in assembly and facilitate manufacturing. Such can be accomplished based upon molding or customized 3D printing. The system and method can include electrodes that apply predetermined electrical signals to the peripheral nerve. Illustratively, the chamber defines an internal surface that is, at least in part, a football, cube, cylinder, semi-sphere, oblong box, or polyhedron.

In an illustrative embodiment method for repairing a damaged peripheral nerve is provided. The nerve is transected to provide two opposing, clean cut ends. A nerve coupler is applied to the nerve at the ends so that the ends are in close proximity to each other and securing the ends in place with respect to the couple, and the ends are bather with a therapeutic agent for treating the damaged area. The bathing of the ends can entail providing a predetermined flow of a fusogen to a chamber surrounding the ends, and holding the coupler, over time. The ends can be secured to the coupler by applying sutures between the peripheral nerve and attachment locations on the coupler. The transecting action can include cutting the nerve at a damaged location in the region of a slot located centrally on the coupler. The nerve coupler can be applied to the damaged nerve by assembling two halves of the nerve coupler together around the nerve in a friction fit. Likewise, the chamber can be assembled the chamber can be applied to the coupler by assembling two halves of the chamber together around the coupler in a friction fit.

In another illustrative embodiment a system and method for repairing a damaged peripheral nerve includes, providing a coupler defining a stabilizing assembly applied to a distal end of a damaged area of the damaged peripheral nerve and applied to a proximal end of the damaged area of the damaged peripheral nerve, the coupler adapted so that the proximal end and the distal end are maintained in a stabile abutting relationship in which nerve repair can occur. A chamber is placed in proximity to the damaged area, and is arranged for containing a predetermined volume of a therapeutic agent for treating the damaged area. Illustratively, the distal end and the proximal end are severed from each other. The coupler can define one of (a) coupler halves that surround the proximal end and the distal end and allow fixing members to be applied to the proximal end and the distal end and (b) cuffs that engage, and are fixed to, each of the proximal end and the distal end and are engaged by overlying coupler halves.

### BRIEF DESCRIPTION OF THE DRAWINGS

The invention description below refers to the accompanying drawings, of which:

FIG. 1 is a fragmentary perspective view of an exemplary peripheral nerve and associated anatomy thereof, for which damage can be treated according to the system and method herein;

FIG. 2 is an exploded, fragmentary perspective view of an exemplary cuff for application to each of the proximal and distal end of a severed peripheral nerve for use with the system and method herein:

FIG. 3 is an exploded, fragmentary perspective view of a nerve coupler system including attached cuffs on the proximal and distal severed peripheral nerve of FIG. 2 and a central coupler with chamber for applying fusogens and/or other agents, or appropriate electrodes, for treating received ends of the severed peripheral nerve and associated cuffs:

FIG. 4 is a fragmentary perspective view of the assembled cuffs and coupler of FIG. 3:

FIG. 5 is a side cross section showing the assembled cuffs and coupler of for receiving the opposing cuffs of FIG. 4, showing the chamber filled with an exemplary fusogen surrounding the damaged nerve ends in stable apposition with respect to each other:

FIG. 6 is a fragmentary, partial side cross section of a severed end of a peripheral nerve and confronting cuff that employs a set of barbs or scales along an inner surface thereof for longitudinally securing the nerve with respect to the cuff, according to an exemplary embodiment:

FIG. 7 is a fragmentary side view of a damaged nerve and associated cuffs and couplers used to secure a graft between severed distal and proximal ends of a damaged peripheral nerve:

FIG. 8 is an exploded, fragmentary side view of a system for repairing a damaged nerve that can be continuous, employing clamshell-like coupler and cuff halves according to an illustrative embodiment:

FIG. 9 is a side view of a split coupler assembly having a plurality of electrical leads and associated electrodes for providing predetermined electrical signals to a damaged nerve:

FIG. 10 is a perspective view of a nerve coupler with a surrounding two-piece fusogen storage chamber according to another illustrative embodiment:

FIG. 11 is a lid for the nerve coupler and fusogen chamber of FIG. 10;

FIG. 12 is a side view of on half of the nerve coupler of FIG. 10 showing the interior thereof:

FIG. 13 is a side view of one half of the nerve coupler of FIG. 10 showing the exterior thereof:

FIG. 14 is a perspective view of one half of the nerve coupler of FIG. 10;

FIGS. 15 and 16 are a front view and a rear view, respectively, of one half of the nerve coupler of FIG. 10;

FIGS. 17 and 18 are each of two disassembled halves of the two-piece fusogen chamber of FIG. 10;

FIG. 19 is a side view of one of the two fusogen chamber halves of FIGS. 17 and 18:

FIGS. 20 and 20A are a front view and a rear view, respectively, of one of the two fusogen chamber halves of FIG. 17:

FIG. 21 is a series of images showing a procedure for transecting a nerve mounted in the nerve coupler of FIG. 10 for subsequent reattachment:

FIG. 22 is an image showing attachment of the nerve coupler of FIG. 10 to a transected nerve to reattach the ends together; and

FIG. 23 is an image of the attached nerve coupler as shown in FIG. 22 with fusogen storage chamber ready for placement therearound.

### DETAILED DESCRIPTION

### I. General Overview

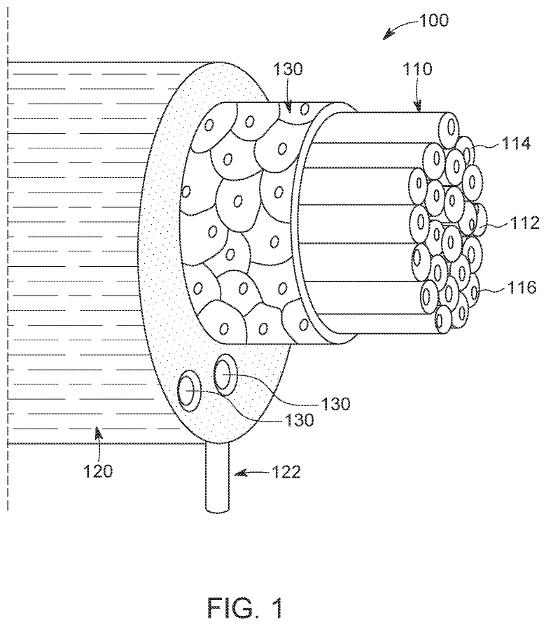

FIG. 1 shows the cellular schematic of an exemplary peripheral nerve trunk 100 that can be fused in accordance with the system and method herein, in which intrafascicular tissue contains the endoneurium 110, Schwann cells 112, and myelin sheath 114 and axon (116) complex. The extrafascicular tissue contains the epineurium 120 and mesoneurium 122; and perineurium 130 lies at the junction of intrafascicular and extrafascicular tissues. Note that the perineurium is the extension of the brain's brain machine interface, and the perineurium is viewed as an extension of the blood-brain interface (BBB). This is a key barrier to re-establish and the illustrative coupler/device of the system and method (described below) can advantageously serve to provide a temporary BBB until the body re-establishes a new one. The nerve is supplied by multiple blood vessels 130. When a nerve is severed, a calcium wave signals the mitochondria in the distal nerve to become permeable. As described generally above, this increased permeability then releases bio factors from the mitochondria of the cell to trigger apoptosis, or programmed cellular axon death, and Wallerian degeneration is initiated by the supportive Schwann cells that surround the distal axons as shown in FIG. 1. The coupler/device herein, hence serves to block apoptosis.

The Schwann cells then recruit monocytes that are stimulated to create macrophages, which, in turn, consume debris from the distal axons, and if the reconnection between severed nerve ends is delayed for a sufficient time period, then the Schwann cells lose their ability to regenerate and support the distal nerve. Hence, delayed attempts to repair the distal nerve will be unsuccessful, and conversely, early reconnection, before the cells degenerate, is critical to full repair.

It has been determined that fusogens, such as PEG, when delivered to the repair site, can both institute fusion of the proximal and distal axons and can also block the mitochondria from signaling. The result would render the distal exons essentially immortal, and thereby prevent Wallerian degeneration thereto. A similar process occurs for a small distance within the proximal nerve stump. This can be termed retrograde degeneration. Such retrograde degeneration can, likewise, be prevented by application of a fusogen (e.g. PEG) to the injury site. It is recognized herein that the use of a physical nerve end coupler is highly desirable to provide a level of stable apposition needed to facilitate fusion in association with application of a fusogen. The illustrative embodiments herein, thus provide both a coupling and a fusogen delivery function in a novel manner.

### II. Nerve Coupler

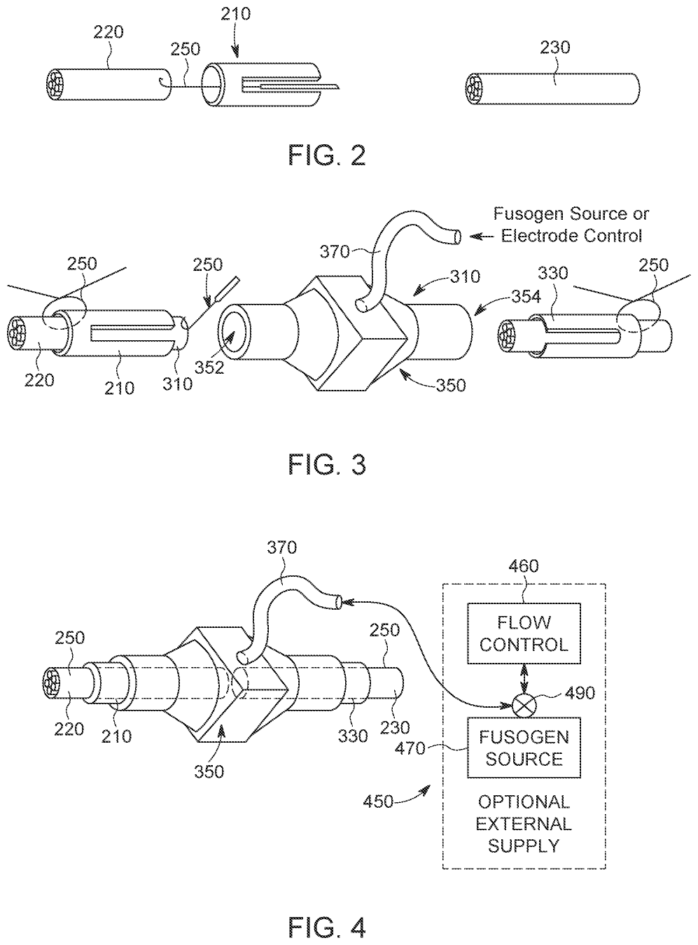

Reference is made to FIGS. 2-4 that show a general example of a nerve coupler 310 according to an embodiment. The depicted nerve coupler 310 consists of at least three parts, including a cuff 210 that is applied to the proximal nerve stump 220. As shown in FIG. 3, the cuff 210 holds the proximal nerve stump 220 securely with the end 310 of the nerve 220 slightly protrudes out of the cuff 210. The cuff 210 is constructed from (e.g.) semipermeable material to allow the nutrients to reach the nerve 220. The material's pore size is small enough to prevent growth of cells through the wall of the cuff 210. The opposing, distal nerve stump 230 is likewise fitted with a second component, consisting of a similar cuff 330 (FIG. 3) as shown to hold it firmly with appropriate permeability. The nerves 220, 230 are secured relative to the cuffs 210, 330 (respectively) using a plurality of possible securing mechanisms. For example, a micro suture 250 can be used. As shown in FIG. 2, the suture 250 is secured through the nerve sheath, and draws the nerve stump into the cuff. The suture 250 is then tied to secure the nerve and cuff together as shown in FIG. 250. The third component, comprising a central coupler 350 (FIG. 3), includes opposing, open ends/sockets 352 and 354 (also termed “apertures”), that are adapted to receive the distal and proximal cuffs 210, 330, respectively. This is shown in FIG. 4, and as shown, the two cuffs 210, 330 can be securely placed into the coupler 350 form opposing proximal and distal ends. The coupler then serves to hold the two severed nerve ends in close apposition. In this manner, the axons of the severed nerve ends are placed in direct, closely proximate and stable apposition and held there for the needed duration. In addition to Marshall, as described above, another example of a closely fitting, cuff-like device, which is placed over a severed nerve ending, and is used for electrical interface, is shown and described in U.S. Pat. No. 9,044,347, entitled HYBRID BIOELECTRICAL DEVICE, issued Jun. 2, 2015, by Paul S. Cederna, et al. However, this cuff includes a closed end that effaces the end of the nerve and provides a neural-electrical interface (in the manner of a plug with respect to a multi-pin socket), rather than a stabilization function relative to an opposed, severed nerve ending.

Note that the coupler/device 310 of this, and other, embodiment(s) herein can be made semi-permeable. Semi-permeability can be accomplished, according to skill in the art, by constructing the material in a braided or similar manner from appropriate fibers. The permeability of a material, such as tyrosine polycarbonate, is controlled by the tightness of the braiding. Tyrosine polycarbonate is an example of various biocompatible materials for use herein, which can be made semi-one such material that can be braided and used to construct the nerve coupler(s) described herein. The tyrosine polycarbonate can be manufactured with nanoparticles incorporated, which carry nerve growth factors for promoting healing of that nerve and also block pain. Additionally, the nanoparticles can attract nerve growth factors.

Optionally, appropriate lines of stem cells (IPSC) can be incorporated in the interstices of the coupler wall, and can thereby transform into nerve cells and other supportive cells. The will block pain and repair the site and act on the procomale nerve to block pain and even in the spinal cord to help to block pain.

Notably, the coupler/device can uniquely bring the proximal and distal ends of the nerve with close approdiations. That is, it can function to place the opposing nerve axons within micrometers of each other to allow fusion of the membranes in a stable manner. It is highly desirable for the axons to fuse end-to-end, side-to-side and membrane-to-membrane so as to maximize the number of axons that successfully fuse.

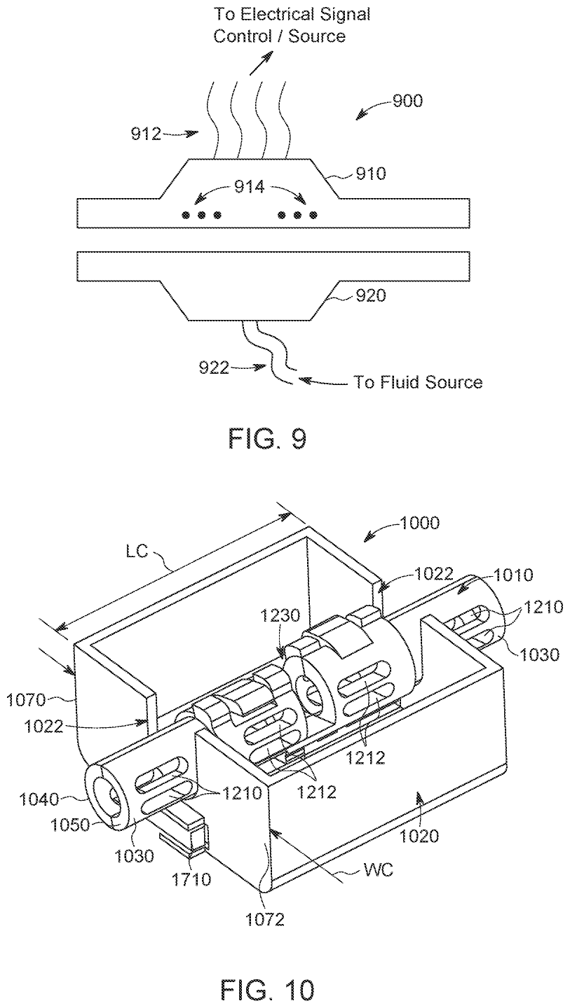

According to an alternate implementation, and as further shown, and described further below, the fusogen conduit 370 which carries fusogen and/or other therapeutic fluids to the damaged nerve can be substituted with, or supplemented with, electrical leads that are interconnected with a controller (see FIG. 9) and that communicate with contacting electrodes that reside within the coupler in contact with various locations on the damaged nerve. The electrodes can operate as stimulators in various embodiments.

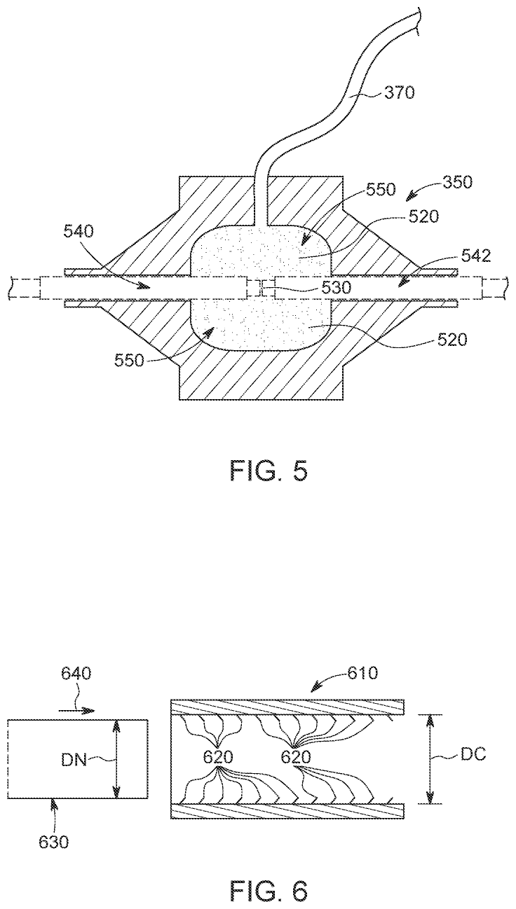

Notably, as shown further in FIG. 5, the inner cavity or chamber 520 of the coupler 350 contains sufficient volume in an area surrounding the break 530 between nerve ends (and associated cuffs) 540 and 542 for a continuous supply of fusogen (and/or other bio agents—shaded component 550) to provide a continuous application of such fusogen to the confronting ends. The fusogen can be supplied to the chamber 520 in advance of the coupling procedure in sufficient amount to support fusion, and/or can be applied subsequent to the coupling. The supply can be delivered via a continuous (e.g. gravity or pump-driven mechanism 450 with flow control 460 from an external (subcutaneous or transcutaneous) source 470, or from a single-use source, such as a syringe. A syringe can be placed through a self-sealing port in the side of the coupler that communicates with the chamber 520. The chamber 520 can also/alternately include a gel or other biocompatible medium that allows fusogen to remain in place during the coupling process, and releases at a timed rate thereafter. As described further below, the volume and length of the chamber is highly variable and can be smaller and/or shorter than that depicted, so long as it allows adequate delivery of fusogen to the damaged region of the nerve. Moreover, in various embodiments, chamber can store stem cells (such as fat cells) that have the ability to increase regeneration and reduce pain, and also help to reduce scar formation. The fat can be harvested from multiple donor sites, which are commonly found, for example, in the abdomen.

Where an active delivery mechanism 450 is employed, the pump 490, which is connected to the flow control 460 can define any acceptable mass-flow device, such as an osmotic pump that interoperates with the flow control, and any associated programming and circuitry, to deliver chemicals over a period time at a specific amount to the chamber.

### III. Additional Features

The surface of the cuffs 210, 350, on both outside and inside surfaces thereof, can be treated so that when a nerve fascicle is pulled into the cuff it will stay in place with the friction of the inner surface of the cuff on the proximal and distal nerve stumps, or as shown in FIG. 6, the inner surface of the exemplary cuff 610 can include a series of directional barbs or scales 620 that can be similar to commercially available barbs/scales on sutures and/or other invasive medical devices. These barbs/scales 620 serve to resist pullout of the nerve 630 once it is inserted (arrow 640) of the nerve, once placed. The cuff 610 inner diameter DC is approximately equal to, or slightly greater than, the outer diameter DN of the nerve 630.

In various embodiments, the outer surface of the cuff(s) can be treated to that when they are put inside either end of the coupler they lock in place against the treated inside surface of the couplers proximal and distal ends.

While the depicted coupler above shows a generally rectilinear outer shape, the chamber (520 above) in the nerve coupler can be provided in a variety of different geometries and can be available in differing sizes and shapes used to accommodate larger nerves with multiple fascicles. The chamber can also be customized to optimize the capacity of the nerve coupler to deliver chemicals to the repair site for a desired time interval and quantity. The chamber shape can be (e.g.) a football (American-style) shape, cube, cylinder, semi-sphere, oblong box, polyhedron, etc. The depicted cross section of the chamber 520 (FIG. 5) is similar at all rotational orientations about the longitudinal axis. In alternate embodiments, the chamber can be dissimilar at varying rotations about that axis.

The coupler chamber can further define a relatively small volume so long as it effectively encompasses the damaged region of the nerve, and allows for a continuous bath to be delivered to the perimeter of the repair region is maintained via a remote source (implanted or external), which can be fed to the chamber via a conduit and (e.g. osmotic) pump. The coupler chamber is also kept relatively small to reduce the need for angiogenesis and so that the eccles are kept close to their supply of oxygen and nutrients. Likewise, the cuffs should fit relatively snugly with respect to the opposing, receiving ends/apertures of the coupler so as to maintain a relatively stable/motion-free apposition between severed nerve ends.

The material for the nerve coupler and/or cuffs can be a permanent material and/or a resorbable material, including, but not limited to, polylactic acid, polyglycolic acid, tyrosine polycarbonate, polycaproic acid, and/or polycaprolactone. The material can be tuned to provide a predetermined resorption time, which can be controlled depending on the need, given the projected healing time, and can range from very short to very long.

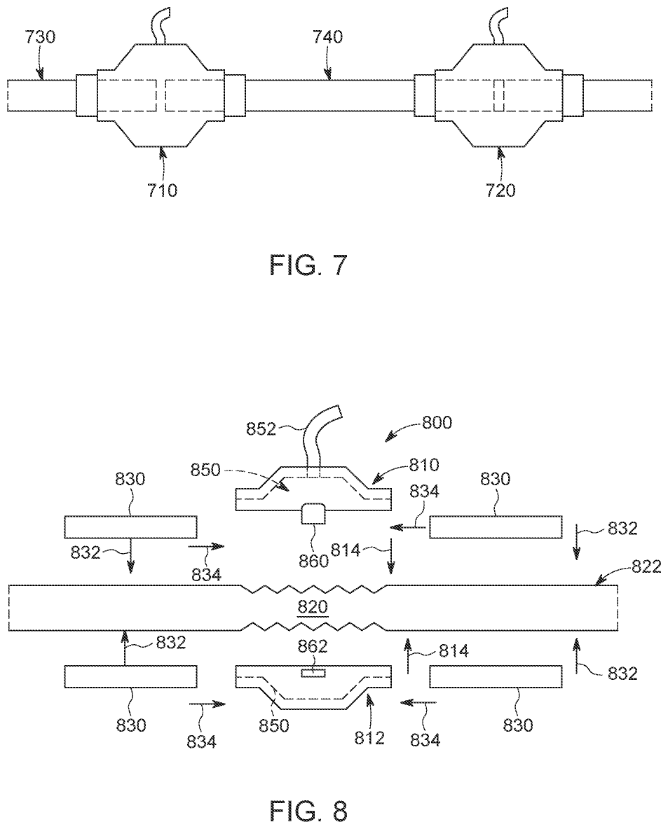

With reference to FIG. 7, it is expressly contemplated that a plurality of nerve couplers 710 and 720 can be employed if there is a gap in the nerve 730 and a living nerve graft 740 needs to be introduced therebetween. Typically, such a living nerve graft is harvested from another region of the patient's own body or another compatible donor. Hence, in the depicted examples, the overall nerve coupler system includes a first couplet 710 at the proximal end of the nerve graft at the proximal repair site and another coupler 720 at the distal nerve site as well. The size, shape and/or performance characteristics (permanence/resorbability, chemical/bioagent delivery, etc. can vary between each coupler 710, 720 in the system.

It is contemplated that the coupler can be adapted for application to a nerve that has been damaged, but remains continuous (not severed/unbroken). As shown in FIG. 8, the coupler assembly 800 can define a pair of engageable halves 810, 812 of appropriate size and shape, which are applied (arrows 814) around the damaged area 820 of the otherwise continuous nerve 822, with or without associated cuff halves 830. The cuff halves can be applied (arrows 832) on each side (proximal/distal) of the damaged area 820 at a location along the nerve length (arrows 834) that overlaps the coupler halves 810, 812 and prevents excess leakage of appropriate agents and chemicals from the chamber region 850 (shown in phantom). The chamber 850 can be sealed, or open to receive chemicals via an inlet with associated feed line 852 from a source, as described above. The coupler halves 810, 812 and/or cuff halves 830 can be held together, once applied, using any external, integral or unitary fastening assembly. For example, the depicted top half 810 can include plastically deformable (spring-loaded) tabs 860 and the bottom half 812 can include conforming detents that receive projections of the tabs in a locking engagement. Alternatively, external sutures can be employed to tie the halves together and/or to the surface of the nerve 822.

In the arrangement of FIG. 8, where the nerve 822 remains continuous, but damaged, the halves can facilitate delivery of regeneration bioagents, such as nerve growth factors and other growth factors to the site of injury, or in some examples, stem cells to the injury site like fat cells that contain a subpopulation of mesenchymal stem cells.

The cuffs and/or coupler can be constructed from a material that can be solid with mechanically bored or molded pores, or can be braided to render it porous so that nutrients can pass through its walls but it still prevents cells like fibroblasts from growing into the repair site and interfering with regeneration. The material can also be made porous by creating small nanoscale perforations in the wall again to allow nutrients and oxygen to the nerves without allowing fibroblasts or other cell types like monocytes or macrophages from interfering with repair and regeneration

Illustratively, the substance of the cuffs or walls of the coupler can have nanospheres attached to it that can either encourage regeneration if directly against the nerve or discourage scar formation if on the outside of the cuff and/or nerve coupler.

According to a further embodiment, the nerve coupler herein can include appropriate connections and other structures to connect an array of electrodes to a. nerve in engagement therewith. Such interconnection can be made in a manner clear to those of skill. In various embodiments, the coupler can be split as described above so as to be applicable to an intact (unsevered) nerve for treatment thereof. By way of example, a coupler 900, arranged (optionally) in the split halves 910 and 920 is depicted in FIG. 9. At least one coupler half 910 (and potentially both halves 910, 920) includes appropriately sized electrical leads 912 that can be loose or bundled into a cable. These leads 912 interconnect with an internal (to the body), external, or both internal/external controller arrangement that provides appropriate signals, and potentially receives sensor data via nerve-engaging electrodes 914. The placement and number of electrodes is highly variable and such can be provided to one or both halves. Similarly this arrangement can be provided to an unsplit coupler as described herein for use with severed nerves. The electrodes can also be provided to sleeves (with appropriate chases to direct leads away from the coupler) rather than the main body of the coupler. At least one coupler half has a fluid conduit 922 for providing/bathing the damaged nerve with a fluid source (fusogens, stem cells (described below)). Hence the coupler 900 can define an appropriately sized/shaped chamber for bathing the nerve in therapeutic fluid. In various embodiments the couple can be free of a fluid source and/or associated chamber, and primarily employed as a stable platform for electrodes.

Note that the depicted coupler, 900, which is split longitudinally, can be used for nerve compression in which each bivalved half is placed around the nerve, and serves in either treatment a for regeneration, or a peripheral stimulator role. In this manner each half can be applied like a half of a cylinder around the nerve to help with regeneration, to reduce pain and/or to block nerve pain with the electrode(s) 912, 914.

By way of example, the coupler can be adapted for use to control pain. The coupler can be used to treat a neuroma in certain applications.

In various embodiments, the coupler can also be employed in a treatment method implicating the application of stem cells to a damaged or diseased nerve. Such stem cells can be provided in fluid suspension after harvesting from an appropriate source-such as the processing of fat by liposuction from the abdominal wall just below the skin of (e.g.) a rat or other mammal. The stem cells would then be processed in a clinical setting (e.g. the operating room). This treatment can assist in relieving a painful nerve. It assists with regeneration of the nerve as it cushions and protects the nerve. Significantly, this technique can act at the repair site or injury site along the proximal nerve and cell body and in the spinal cord to block pain.

### IV. Nerve Coupler Friction-Fit Halves and Transection Slot

### A. Mechanical Design

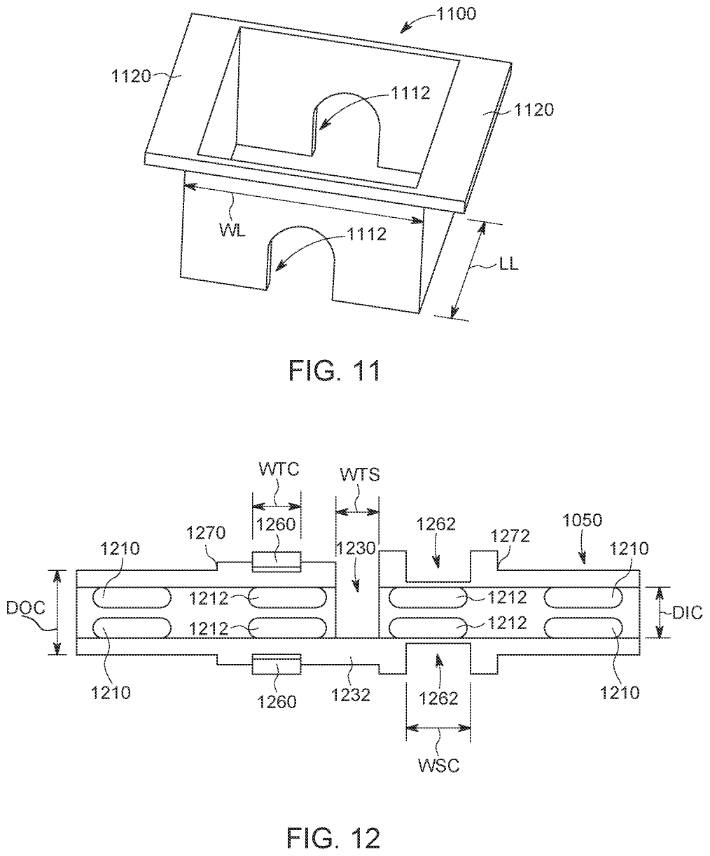

FIG. 10 shows a further illustrative embodiment of a nerve coupler arrangement 1000. The arrangement 1000 is adapted to retain two severed nerve ends in a stable, closely confronting orientation as described generally above so that fusogen, such as PEG can remain in contact with the ends during reconnection. The arrangement comprises a two piece nerve coupler 1010 that is cradled within a two-piece fusogen storage chamber (also termed a reservoir) 1020. The chamber includes cutouts on each of opposing ends to receive the opposing (cylindrical) distal ends 1030 of the coupler 1010. The bottoms of each respective chamber cutout 1022 are semicircular to conform to the shape of each cylindrical distal end of the cutout. In operation, the chamber is filled with fusogen to thereby bathe the nerve ends with fusogen. A lid 1100 (FIG. 11) is seated over the chamber, and incudes corresponding semi-circular-bottomed cutouts 1112 that secure the coupler 1010 in place and cover the chamber cutouts 1022, when assembled, to reduce fusogen leakage from the chamber.

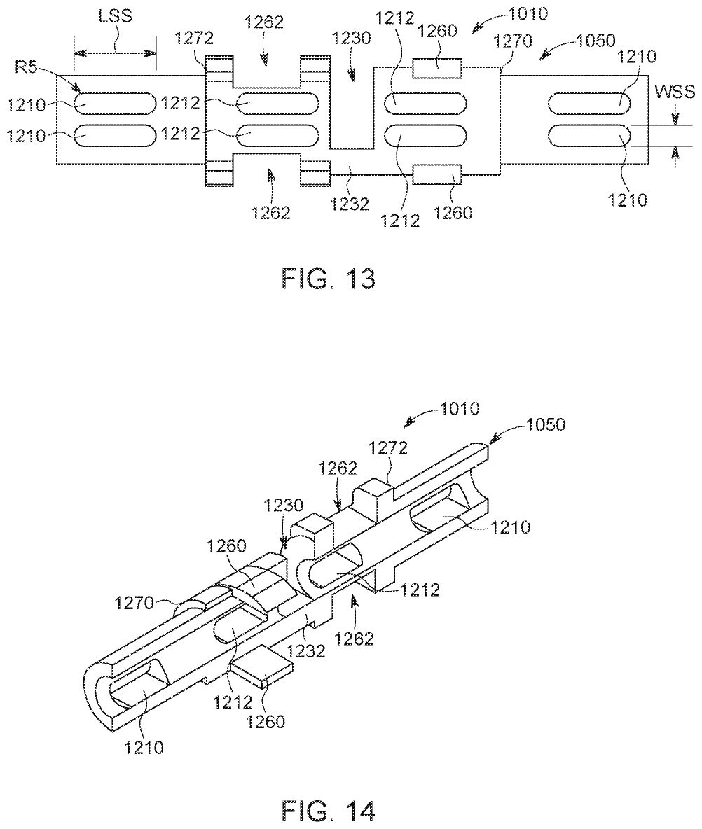

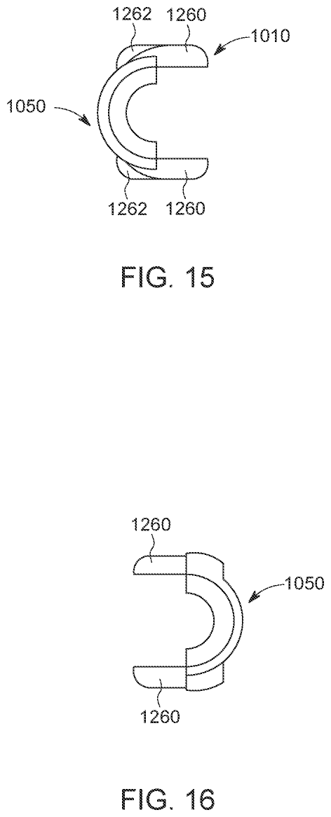

In general, the mechanical design of the coupler 1010, chamber/reservoir 1020, and lid 1100 defines a slim cylindrical shell with internal geometry that allows for temporary exposure to fusogens while also securing proximal and distal ends from unwanted movement. With further reference to FIGS. 12-16, the coupler 1010, peripheral 1210 and central suture 1212 slots to allow surgeons to secure the nerve ends and help prevent retraction, rotation, and tension of the nerve post transection. The slot design is compatible with curved micro-suturing tools and its dimensions were optimized for these tools. The use of both peripheral and central slots allows for easy access to the nerve on multiple ends and gives freedom to surgeons to use their preferred suturing technique. The central section of the coupler 1010 also includes a transection gap 1230 and a bottom support 1232. Together, these components allow for the in-vivo transection of the nerve (described below), and gives the surgeon a flat surface to utilize as a cutting board. Following transection, the bottom support 1232 aids in maintaining stability of the nerve and prevents it from drooping. The transection gap 1230 also functions as the primary location for the exposure of the nerve ends to fusogen.

The coupler is formed in two identical halves (one half 1050) shown in FIGS. 12-16) that fit together in only one orientation in a clamshell arrangement. This can help to reduce/eliminate confusion by the surgeon as to the orientation of the device during implantation. Manufacturing and future design iterations also benefit from this design given that one part being provided, as opposed to two distinct parts each being molded or printed once. The coupler halves 1040 and 1050 of the coupler 1010 each have friction fit attachment structures that slide together to house the nerve ends. In an embodiment, one side of the coupler 1010 (relative to slot 1230) includes a pair of diametrically opposed tabs 1260 and the opposing side contains a pair of receiving slots 1262. The depicted, friction fit design is also reversible as a surgeon will be able to detach one half 1040 from the other half 1050 in the event of coupler adjustment or removal.

In general, the width WTC of each tab 1260 is equal to or slightly less than the width WSC of each slot 1262. More general, the dimensions, herein, such as coupler inner diameter DIC along its inner lumen, outer diameter DOC, slot length LSS, width WSS and slot end radius RS are selected to provide appropriate mechanical strength within the relative scale of the nerve being treated. These dimension are, thus, highly variable and scaled to the size of the nerve. In practice, the coupler 1010, chamber 1020 and lid 1100 can be manufactured in a variety of fixed sizes, or alternatively, can be constructed to custom dimensions based on the diameter and/or other parameters of the nerve or treatment site. By way of non-limiting example, a custom version can be constructed using computer-processor-controlled 3D printing techniques. Such techniques can be carried out using hardware and software known to those of skill in the art. One material that can be employed for such 3D printing is UV-cured BioClear resin available from FormLabs, Inc. of Somerville, MA. A variety of other non-porous and/or porous materials can be employed for various components of the system, including resorbable materials as described above.

The inner lumen diameter DIC of the coupler can, thereby, be chosen to conform to the diameter of the nerve being treated and other dimensions are appropriately scaled. This can be accomplished using a computer processor via appropriate scaling algorithms. The overall structure can remain low in profile according to such algorithms by applying appropriate parameters that account for minimum required structural strength of components-so that (e.g.) wall thickness does not increase at the same rate as coupler diameter. Note, for purposes of scaling, and by way of non-limiting example, the coupler inner lumen diameter DIC is approximately 1.8 mm, the outer diameter DOC is approximately 3 millimeters and the overall length is more than 13 millimeters.

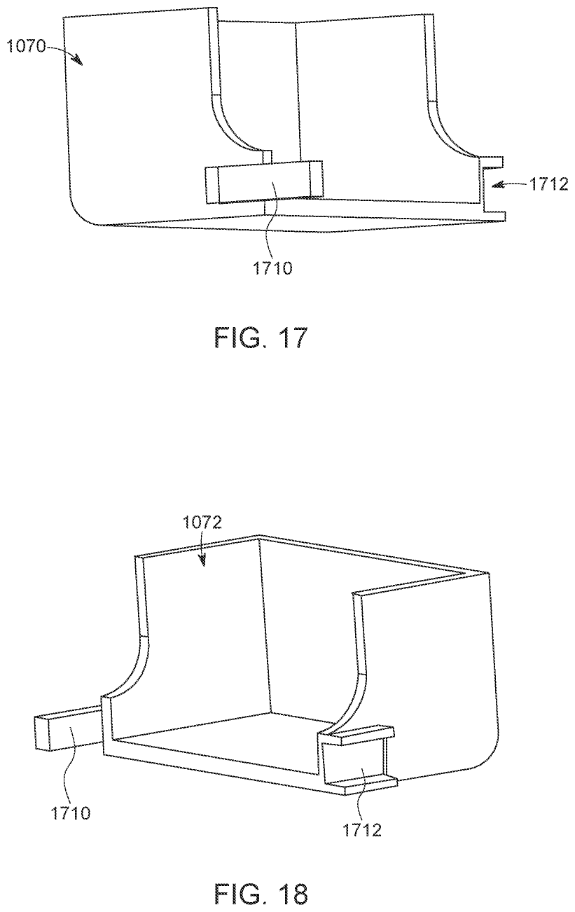

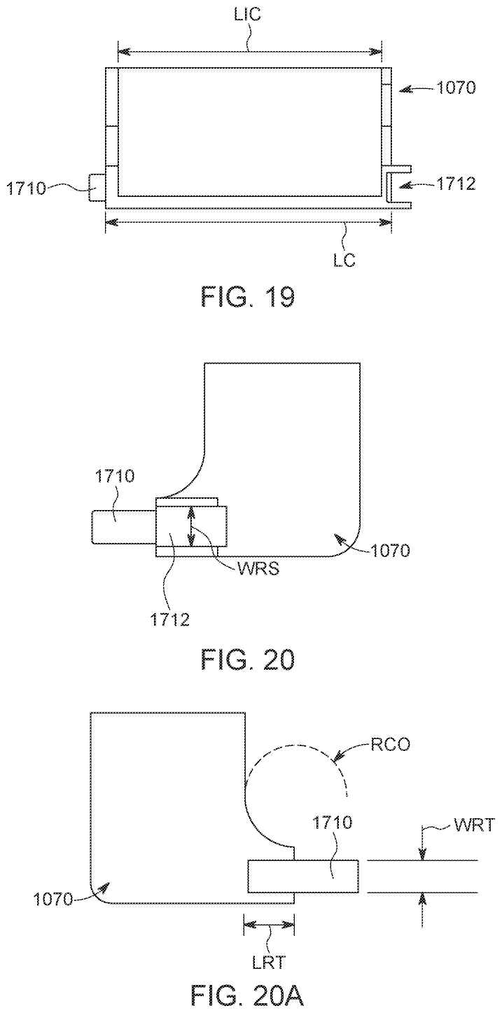

With further reference to FIGS. 17-20A, the chamber/reservoir 1020 and lid 1020 are adapted to collectively (when assembled) house fusogen immediately after transection of the nerve. The width WC and length LC (FIG. 10) of the chamber 1020 is minimized to avoid awkward manipulation within the patient's anatomy. Likewise, the two halves 1070 and 1072 of the chamber 1020 lock together by two bottom-positioned friction fits, enabling the reservoir to be slid under the nerve, and avoid lifting the nerve too high off the surrounding surgical environment (which may otherwise impart undesirable tension on the nerves). The friction fits are provided by opposing tabs 1710 and slots 1712. The length LRT and width WRT of the tabs 1710 are adapted to be received by the slots 1712 having a width WRS that is equal to, or slightly greater than, the tab width WRS. To avoid leakage over the top of the coupler 1010 in the assembled arrangement, once the chamber halves 1070 and 1072 are fit together, the lid 1100 is slid within the walls of the chamber 1020, creating a seal around the coupler. The lid design of this embodiment is adapted for particular purposes, and includes an open top for the direct application and removal of fusogen and side wings 1120 for easy manipulation with microsurgical tools. In alternate embodiments, the lid can be sealed, and/or include ports for feeding/removing/replenishing fusogen after the patient's surgical site is closed. In further embodiments, a two-piece chamber and one-piece lid structure can be replaced with a two piece sealed chamber that is secured around the coupler after it is installed—for example an outer, coaxial cylinder that stores fusogen and can include appropriate ports.

The use two halves of a chamber 1020 with the same geometry, which can only be assembled in one direction has the same benefits as the coupler in that it simplifies molding or 3D printing and avoids confusion during surgical placement. Both the chamber and the lid can be provided in fixed sizes of custom printed with appropriate scaling to match the dimensions of the coupler.

The length between inner walls LIC of the chamber and the corresponding width of the inner walls in the assembled chamber are sized to receive a lid with an outer wall length LL (FIG. 11) and width WL in a manner that is snug but slidable. As described above, both the chamber 1020 and the lid 1100 include respective cutouts 1022 and 1112 that are adapted to cradle and secure the outer ends of the coupler 1010. The inner section defines a larger diameter than the outer sections with opposing a step-down shoulders 1270 and 1272 that are spaced apart along the length of the coupler to reside snugly within the inner walls of bother the chamber and nested lid. As such, the step-down shoulders engage the chamber/lid walls at the cutouts in a manner that limits axial movement between these components and forms a liquid seal for fusogen. The radius RCO (FIG. 20A) of each cutout 1022, 1112 conforms to the outer diameter DOC of each outer end of the coupler 1010 to limit radial movement of the coupler relative to the chamber/lid and enhance the liquid seal.

### B. Operational Use and Surgical Procedure

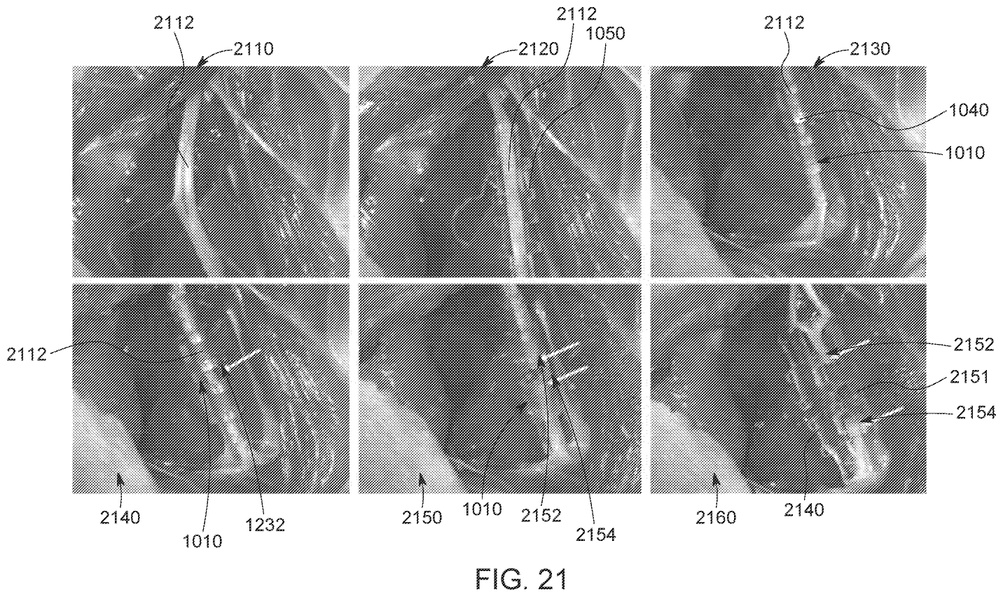

Reference is made to FIG. 21 a series of images depict an exemplary peripheral nerve transection procedure using the slot 1230 of the coupler 1010. In this example, the sciatic nerve of a euthanized rodent is depicted, but the procedures herein are applicable to any living subject, including human patients. The exemplary sciatic nerve 2112 is first exposed in image 2110 exposed via (e.g.) a split-gluteal muscle approach coursing through the popliteal fossa of the left hind leg. The common peroneal and tibial nerve compartments can be visualized as two distinct fascicles of the sciatic nerve 2112. In image 2120, the one half 1050 of the coupler is placed under the sciatic nerve 2112. Then, in image 2130, the other half 1040 of the coupler is placed in the field and attached to the first coupler half 1050 by hand, based on a friction fit between tabs and slots, thereby forming the assembled coupler 1010 around the nerve 2112. In image 2140, the assembled coupler 1010 is rotated such that the cutting board (1232) is under the nerve 2112. Two (e.g.) epineurial 8-0 prolene stay sutures are placed on each end to secure the nerve to the coupler. Then, in image 2150 a sharp transection was made using (e.g.) an 11 blade. There is a small degree of nerve stump retraction between ends 2152 and 2154 in this example, which through proper procedures can be minimized. In image 2160, the two halves 2140 and 2151 of the coupler can be opened by overcoming the friction fit between tabs and slots. In this image, a clean transection can be readily visualized between nerve stumps 2152, 2154. The transected nerve provides two clean ends that a good candidate for fusion, as described below.

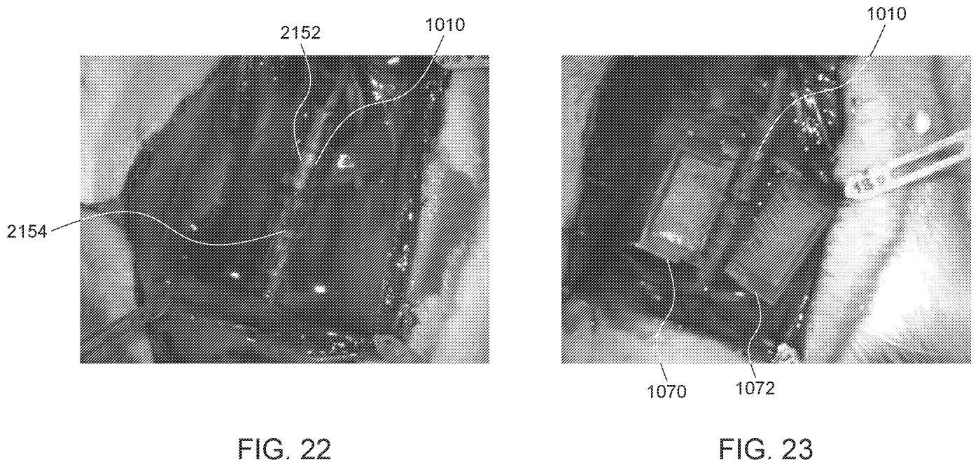

With reference to FIG. 22, the coupler is attached to, and sutured onto, the transected nerve ends 2152 and 2154 in close proximity to each other to afford the best chance of fusion therebetween. The halves of the fusogen chamber/reservoir are then brought into proximity with the attached coupler 1070 and 1072. In a next step (not shown) the chamber halves 1070, 1072 are assembled around the coupler through a friction fit between tabs and slots and the lid is slid over the assembled chamber to lock the coupler in place and allow for introduction of fusogen.

### V. Conclusion

It should be clear that the above-described embodiments for a nerve coupler that allows delivery of appropriate fusogens and/or other bio agents to a severed or damaged nerve provides a highly desirable treatment system and method for such injuries. This arrangement allows for substantially more rapid healing with minimal loss of function in many instances. This system and method can be used for isolated injuries or elongated injuries along a portion of a nerve that may require a graft. The coupler arrangement herein can be permanent or can be resorbable depending upon the requirements. More particularly, the coupler can be constructed from resorbable Marcaine bupivacaine so that when it degrades, it releases long acting anesthetics (embedded in the material by conventional or custom techniques) at the repair site. Such agents can also be embedded as nanoparticles in the material. It is further recognized that application of the nerve coupler according to the various embodiments herein can be accomplished using existing neurosurgical and/or microsurgical techniques and equipment that are performed manually and/or using automated mechanisms (e.g. surgical robots, minimally invasive techniques, imaging systems, etc.). The coupler can apply fusogens, other growth factors, NGF and/or anesthetics, so as to provide long-term anesthesia and relief of pain. Alternatively, or additionally the coupler can act as a stimulator to provide more long term pain relief by over stimulation of the nerve and thereby driving it into its absolute refractory period, in which its sodium potassium pump prevents it from firing additional impulses that cause pain.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments of the apparatus and method of the present invention, what has been described herein is merely illustrative of the application of the principles of the present invention. For example, while the above-described nerve coupler system and method is particularly adapted to the repair of damaged and severed nerve endings, it is expressly contemplated that the combined stabilization and application of therapeutic agents afforded thereby can be applied to other elongated structures within the body, such as vasculature tissue, tendons, and the like, with appropriate modification. Also, while the foregoing describes a number of separate embodiments of the apparatus and method of the present invention, what has been described herein is merely illustrative of the application of the principles of the present invention. For example, also as used herein, various directional and orientational terms (and grammatical variations thereof) such as “vertical”, “horizontal”, “up”, “down”, “bottom”, “top”, “side”, “front”, “rear”, “left”, “right”, “forward”, “rearward”, and the like, are used only as relative conventions and not as absolute orientations with respect to a fixed coordinate system, such as the acting direction of gravity. Additionally, where the term “substantially” or “approximately” is employed with respect to a given measurement, value or characteristic, it refers to a quantity that is within a normal operating range to achieve desired results, but that includes some variability due to inherent inaccuracy and error within the allowed tolerances (e.g. 1-2%) of the system. Note also, as used herein the terms “process” and/or “processor” should be taken broadly to include a variety of electronic hardware and/or software based functions and components. Moreover, a depicted process or processor can be combined with other processes and/or processors or divided into various sub-processes or processors. Such sub-processes and/or sub-processors can be variously combined according to embodiments herein. Likewise, it is expressly contemplated that any function, process and/or processor herein can be implemented using electronic hardware, software consisting of a non-transitory computer-readable medium of program instructions, or a combination of hardware and software. Accordingly, this description is meant to be taken only by way of example, and not to otherwise limit the scope of this invention.

What is claimed is:

1. A system for repairing a damaged peripheral nerve comprising:
a coupler defining a first coupler half and a second coupler half divided along a longitudinal plane having an inner lumen that is sized and arranged to receive each side of the peripheral nerve at a transected location thereof, the coupler including slots for receiving sutures that anchor each side in an abutting orientation; and
a chamber adapted to surround a portion of the coupler so as to bathe the transected location in a therapeutic agent.

2. The system as set forth in claim 1 wherein the coupler includes a slot and bottom support located centrally along the longitudinal plane constructed an arranged to facilitate transection of the peripheral nerve, the bottom support being arranged to maintain the coupler free of flexure.

3. The system as set forth in claim 1 wherein the first coupler half and the second coupler half each include interengaging, friction fit tabs and slots for removably securing one to the other.

4. The system as set forth in claim 3 wherein the first coupler half and the second coupler half are substantially identical in size and shape.

5. The system as set forth in claim 1 wherein the coupler or the chamber is constructed from at least one of permanent and resorbable materials.

6. The system as set forth in claim 1, further comprising, an external source of the therapeutic agent that flows to the chamber at a predetermined rate.

7. The system as set forth in claim 6, further comprising, a flow controller that meters the rate.

8. The system as set forth in claim 1 wherein the therapeutic agent is a fusogen.

9. The system as set forth in claim 8 wherein the fusogen comprises at least one of PEG, chitosan, dextran sulfate, n-nonyl bromide, calcium, sodium nitrate, and H-α-7.

10. The system as set forth in claim 1 wherein the first chamber half and the second chamber half each include interengaging, friction fit tabs and slots for removably securing one to the other.

11. The system as set forth in claim 10 further comprising a lid adapted to nest within inner walls of the chamber and surround the coupler.

12. The system as set forth in claim 11 wherein the first chamber half and the second chamber half are substantially identical in size and shape.

13. The system as set forth in claim 1, further comprising, electrodes that apply predetermined electrical signals to the peripheral nerve.

14. The system as set forth in claim 1 wherein the chamber defines an internal surface that is, at least in part, a football, cube, cylinder, semi-sphere, oblong box, or polyhedron.

15. A method for repairing a damaged peripheral nerve comprising the steps of:
transecting the nerve to provide two opposing, clean cut ends;
applying a nerve coupler to the nerve at the ends so that the ends are in close proximity to each other and securing the ends in place with respect to the coupler; and
bathing the ends with the therapeutic agent for treating the damaged area.

16. The method as set forth in claim 15 wherein the step of bathing comprises directing a predetermined flow of a fusogen to a chamber surrounding the ends, and holding the coupler, over time.

17. The method as set forth in claim 15 wherein the step of securing includes applying sutures between the peripheral nerve and attachment locations on the coupler.

18. The method as set forth in claim 17 wherein the step of transecting includes cutting the nerve at a damaged location in the region of a slot located centrally on the coupler.

19. The method as set forth in claim 17 wherein the step of applying the nerve coupler includes assembling two halves of the nerve coupler together around the nerve in a friction fit.

20. The method as set forth in claim 19 wherein the step of applying the chamber includes assembling two halves of the chamber together around the coupler in a friction fit.

21. A system for repairing a damaged peripheral nerve comprising:
a coupler defining a stabilizing assembly applied to a distal end of a damaged area of the damaged peripheral nerve and applied to a proximal end of the damaged area of the damaged peripheral nerve, the coupler adapted so that the proximal end and the distal end are maintained in a stabile abutting relationship in which nerve repair can occur; and
a chamber in proximity to the damaged area, arranged for containing a predetermined volume of a therapeutic agent for treating the damaged area.

22. The system as set forth in claim 21 wherein the distal end and the proximal end are severed from each other.

23. The system as set forth in claim 21 wherein the coupler defines one of (a) coupler halves that surround the proximal end and the distal end and allow fixing members to be applied to the proximal end and the distal end and (b) cuffs that engage, and are fixed to, each of the proximal end and the distal end and are engaged by overlying coupler halves.

24. The system as set forth in claim 21 wherein the coupler halves or the cuffs are constructed from at least one of permanent and resorbable materials.

25. The system as set forth in claim 21, further comprising, an external source of the therapeutic agent that flows to the chamber at a predetermined rate.

26. The system as set forth in claim 25, further comprising, a flow controller that meters the rate.

27. The system as set forth in claim 21 wherein the damaged area is a severed nerve and the therapeutic agent is a fusogen.

28. The system as set forth in claim 27 wherein the fusogen comprises at least one of PEG, chitosan, dextran sulfate, n-nonyl bromide, calcium, sodium nitrate, and H-α-7.

29. The system as set forth in claim 21, wherein the fixing member comprises a coupler that includes pair of coupler halves that are selectively joined together to surround the damaged area.

30. The system as set forth in claim 21 wherein the coupler includes a slot and bottom support located centrally along the longitudinal plane constructed an arranged to facilitate transection of the peripheral nerve, the bottom support being arranged to maintain the coupler free of flexure.

31. The system as set forth in claim 21 wherein the coupler includes electrodes that apply predetermined electrical signals to the peripheral nerve.

32. The system as set forth in claim 21 wherein the chamber defines an internal surface that is, at least in part, a football, cube, cylinder, semi-sphere, oblong box, or polyhedron.
